(19) United States
(12) Patent Application Publication
(10) Pub. No.: US 2023/0118677 A1
(43) Pub. Date: Apr. 20, 2023

(54) STRESS MANAGEMENT APPARATUS, STRESS MANAGEMENT METHOD, AND COMPUTER READABLE RECORDING MEDIUM

(71) Applicant: NEC Corporation, Minato-ku, Tokyo (JP)

(72) Inventor: Tasuku KITADE, Tokyo (JP)

(73) Assignee: NEC Corporation, Minato-ku, Tokyo (JP)

(21) Appl. No.: 17/911,761

(22) PCT Filed: Mar. 27, 2020
(86) PCT No.: PCT/JP2020/013980

§ 371 Date: Sep. 15, 2022

(51) Int. Cl. A61B 5/16; G16H 50/30; G16H 10/60

(52) U.S. Cl. CPC A61B 5/165; G16H 50/30; G16H 10/60

(57) ABSTRACT

The stress management apparatus includes: a stress level calculation unit that calculates a stress level of a target subject based on physiological information of the target subject; a person specification unit that specifies, based on an activity history of the target subject, a person involved in an activity that has caused stress of the calculated stress level to be incurred; an influence degree calculation unit that calculates, based on the calculated stress level, an influence degree indicating a degree of influence of the specified person on stress given to the target subject; and an output unit outputs management information based on the calculated influence degree.

### TECHNICAL FIELD

The present invention relates to a stress management apparatus and a stress management apparatus for managing stress that a person gives to their surroundings, and further relates to a computer readable recording medium that includes a program recorded thereon, the program being intended to realize them.

### BACKGROUND ART

Normally, various types of external stimuli place stress on a person. Also, among different types of stress, especially stress that is caused in connection with labor is called occupational stress. Occupational stress often leads to the development of a mental disorder, such as clinical depression, and further causes reduced productivity, separation from work, a leave of absence from work, and so on.

Examples of major causes of occupational stress include interpersonal relationships at work. In order to reduce stress caused by interpersonal relationships, it is important that stress on a person be recognized not only by the side who has been subjected to the stress, but also by the side who gives the stress (stressor) to the other side. For this reason, for example, patent document 1 discloses a system that provides a stressor with a notification indicating that he/she has given stress to another person.

### LIST OF RELATED ART DOCUMENTS

### Patent Document

Patent document 1: Japanese Patent Laid-Open Publication No. 2018-045545

### SUMMARY OF INVENTION

### Problems to be Solved by the Invention

However, according to the system disclosed in patent document 1, a stressor is estimated based on keywords extracted from the result of recognition of sounds in sound data. Therefore, the system disclosed in patent document 1 has a problem that it is difficult to estimate a stressor who gives stress to another person without giving out specific keywords.

An example object of the present invention is to provide a stress management apparatus, a stress management method, and a computer readable recording medium that solve the aforementioned problem and make it possible to estimate even a stressor who gives stress to another person non-verbally.

### Means for Solving the Problems

In order to achieve the above-described object, a stress management apparatus, includes:

a stress level calculation unit that calculates a stress level of a target subject based on physiological information of the target subject;

a person specification unit that specify, based on an activity history of the target subject, a person involved in an activity that has caused stress of the calculated stress level to be incurred;

an influence degree calculation unit that calculates, based on the calculated stress level, an influence degree indicating a degree of influence of the specified person on stress given to the target subject; and

an output unit that outputs management information based on the calculated influence degree.

In addition, in order to achieve the above-described object, a stress management method includes:

a stress level calculation step of calculating a stress level of a target subject based on physiological information of the target subject;

a person specification step of specifying, based on an activity history of the target subject, a person involved in an activity that has caused stress of the calculated stress level to be incurred;

an influence degree calculation step of calculating, based on the calculated stress level, an influence degree indicating a degree of influence of the specified person on stress given to the target subject; and

an output step of outputting management information based on the calculated influence degree.

Furthermore, in order to achieve the above-described object, a computer readable recording medium according to an example aspect of the invention is a computer readable recording medium that includes recorded thereon a program,

the program including instructions that cause the computer to carry out

a stress level calculation step of calculating a stress level of a target subject based on physiological information of the target subject;

a person specification step of specifying, based on an activity history of the target subject, a person involved in an activity that has caused stress of the calculated stress level to be incurred;

an influence degree calculation step of calculating, based on the calculated stress level, an influence degree indicating a degree of influence of the specified person on stress given to the target subject; and

an output step of outputting management information based on the calculated influence degree.

### Advantageous Effects of the Invention

As described above, according to the invention, it is possible to estimate even a stressor who gives stress to another person non-verbally.

### BRIEF DESCRIPTION OF THE DRAWINGS

FIG. 1 is a block diagram illustrating a schematic configuration of the stress management apparatus according to the example embodiment.

FIG. 2 is a block diagram specifically illustrating the configuration of the stress management apparatus according to the example embodiment.

FIG. 3 is a flow diagram illustrating the operations of the stress management apparatus according to the example embodiment.

FIG. 4 is a block diagram illustrating an example of a computer that realizes the stress management apparatus according to the example embodiment.

### EXAMPLE EMBODIMENT

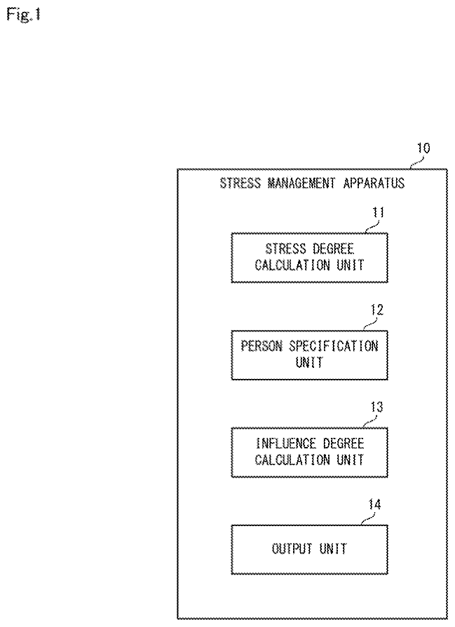

The following describes a stress management apparatus, a stress management method, and a program according to an example embodiment with reference to FIG. 1 to FIG. 4.

[Apparatus Configuration]

First, a schematic configuration of the stress management apparatus according to the example embodiment will be described using FIG. 1. FIG. 1 is a block diagram illustrating a schematic configuration of the stress management apparatus according to the example embodiment.

A stress management apparatus 10 according to the example embodiment illustrated in FIG. 1 is an apparatus for managing stress that a person gives to their surroundings. As illustrated in FIG. 1, the stress management apparatus 10 includes a stress level calculation unit 11, a person specification unit 12, an influence degree calculation unit 13, and an output unit 14.

In this configuration, the stress level calculation unit 11 calculates a stress level of a target subject based on physiological information of the target subject. Based on an activity history of the target subject, the person specification unit 12 specifies a person involved in an activity that has caused stress of the calculated stress level to be incurred.

Based on the calculated stress level, the influence degree calculation unit 13 calculates an influence degree indicating a degree of influence of the specified person on the stress given to the target subject. The output unit 14 outputs management information that is obtained based on the calculated influence degree.

In the example embodiment, a person related to an activity that caused stress on a target subject is specified from an activity history of the target subject, and the influence of this person on the stress is quantified. Therefore, according to the example embodiment, it is possible to estimate a stressor who gives stress to another person non-verbally.

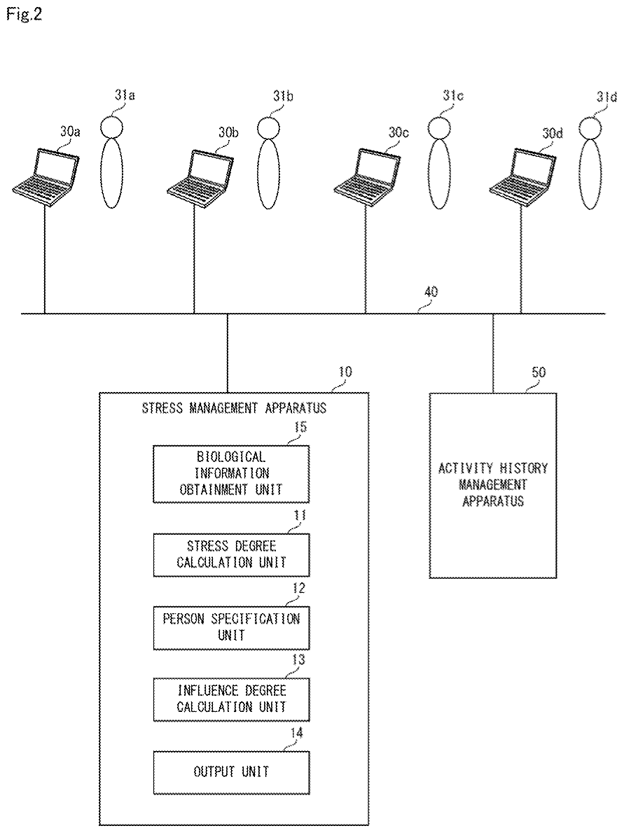

Subsequently, the configuration and functions of the stress management apparatus 10 according to the example embodiment will be specifically described using FIG. 2. FIG. 2 is a block diagram specifically illustrating the configuration of the stress management apparatus according to the example embodiment.

As illustrated in FIG. 2, according to the example embodiment, the stress management apparatus 10 is connected to terminal devices 30a to 30d of respective target subjects 31a to 31d, from which physiological information is obtained, via a network 40 such as a LAN (Local Area Network). The target subjects 31a to 31d, for example, belong to the same group in an organization. In the example embodiment, the number of target subjects is not limited to four, which is exemplarily illustrated in FIG. 2, and any number of target subjects may be present.

Furthermore, as illustrated in FIG. 2, the stress management apparatus 10 is also connected to an activity history management apparatus 50 via the network 40. The activity history management apparatus 50 manages activity histories of the target subjects 31a to 31d.

Here, examples of an activity history include information related to past activities of a target subject, such as information related to electronic mails received or sent by the target subject (e.g., receivers, senders, and documents), and information related to meetings attended by the target subject (e.g., the minutes of the meetings, the list of names of attendees, sound data of the meetings, and seating positions). Also, the activity information management apparatus 50 may be, for example, a mail server or a schedule management server, or may be a combination thereof.

As illustrated in FIG. 2, the stress management apparatus 10 also includes a physiological information obtainment unit 15, in addition to the above-described stress level calculation unit 11, person specification unit 12, influence degree calculation unit 13, and output unit 14.

The physiological information obtainment unit 15 obtains physiological information of each target subject. Specifically, physiological information of a target subject is obtained via a sensor, a wearable terminal, and the like mounted on the body of the target subject, and imported to a terminal device. The physiological information obtainment unit 15 obtains pieces of physiological information of respective target subjects from respective terminal devices 30a to 30d via the network 40.

In the example embodiment, examples of physiological information include a heart rate, an electrodermal potential, an amount of sweating, a facial image, and acceleration indicating a person's movement. Furthermore, physiological information may be only one of these, or may be a combination of two or more of these.

In the example embodiment, the stress level calculation unit 11 calculates a stress level with use of an existing method, and further a method that will be developed in the future, in accordance with the type of physiological information obtained by the physiological information obtainment unit 15. A stress level is a measure of the extent of stress, and also includes the extent of the elevation or decrease in stress based on a certain point in time. For example, in a case where physiological information is a heart rate, the stress level calculation unit 11 calculates a characteristic amount from a chronological change in the heart rate, and converts the calculated characteristic amount into a stress level. Also, in a case where physiological information includes a plurality of types of information, the stress level calculation unit 11 can also estimate a stress level with use of the plurality of types of physiological information.

For example, in a case where physiological information is acceleration indicating a person's movement, examples of a method of calculating a stress level include a method disclosed in the following reference document 1 or 2.
Reference document 1: A. Sano et al., “Recognizing academic performance, sleep quality, stress level, and mental health using personality traits, wearable sensors and mobile phones,” in Wearable and Implantable Body Sensor Networks (BSN), 2015 IEEE 12^{th} International Conference on, 2015, pp. 1-6.
Reference document 2: Yoshiki Nakajima and two others, “Improvement in Chronic Stress Level Recognition by Using Both Full-term and Short-term Measurements of Physiological Features”, The 32nd Annual Conference of the Japanese Society for Artificial Intelligence, 2018.

In the example embodiment, the person specification unit 12 specifies, for each of the target subjects 31a to 31d, an activity that causes stress on the target subject from the activity histories managed in the activity history management apparatus 50, and further specifies a person involved in the specified activity. Also, a person specified at this time may be another target subject who belongs to the same group, or may be a person outside the group.

For example, assume that a target subject received an electronic mail in a time period in which the stress level exceeded a set value. In this case, the person specification unit 12 specifies, from the activity history management apparatus 50, an activity intended for the electronic mail as an activity that causes stress. Then, the person specification unit 12 obtains the electronic mail that serves as the cause, and specifies at least one of the receiver and the sender of the obtained electronic mail and people who appear in the text thereof as a person.

Also, for example, assume that a target subject was attending a meeting in a time period in which the stress level exceeded a set value. In this case, the person specification unit 12 specifies, from the activity history management apparatus 50, an activity intended for the meeting as an activity that causes stress. Then, the person specification unit 12 obtains information related to the specified meeting, and specifies a person involved in the meeting from the obtained information.

Specifically, the person specification unit 12 specifies a speaker by applying known techniques called speaker recognition, speaker verification, and speaker identification to sound data of the meeting. Then, the person specification unit 12 specifies the speaker as a person involved in the activity that causes stress.

Also, in a case where a plurality of people have been specified by the person specification unit 12, the influence degree calculation unit 13 calculates the influence degree for each of the plurality of people based on the stress levels that have been calculated with respect to the target subject.

The influence degree calculation unit 13 can also set a weight for the specified person in accordance with the relationship between the specified person and the target subject, and calculate the influence degree in accordance with the set weight. For example, the influence degree calculation unit 13 sets a relatively small weight in a case where the target subject is a supervisor of the specified person, and sets a relatively large weigh in a case where the target subject is a subordinate of the specified person. Furthermore, the weight may be set in accordance with a person's post or role.

Furthermore, assume that an activity that causes stress is an activity intended for an electronic mail, and the person specification unit 12 has specified at least one of the receiver and the sender of the electronic mail and people who appear in the text thereof. In this case, the influence degree calculation unit 13 sets a weight for the specified person in accordance with the role of the specified person in the electronic mail, and calculates the influence degree in accordance with the set weight.

Furthermore, assume that an activity that causes stress is an activity intended for a meeting, and the person specification unit 12 has specified a person involved in the meeting. In this case, the influence degree calculation unit 13 sets a weight for the specified person in accordance with the behavior of the specified person in the meeting, and calculates the influence degree in accordance with the set weight.

Specifically, the influence degree calculation unit 13 can set a weight with use of a regression model in which various environmental factors (triggers) that cause stress are used as explanatory variables and a stress value is used as an objective function. Then, the influence degree calculation unit 13 can calculate an influence degree from the weight that has been set by the regression model. One example of the regression model is as indicated by the following Math. 1.

$$S\_Yi \_j = f ( X 1 \_j , X 2 \_j , … , Xn\_j ) \qquad ( Math . 1 )$$

$$= w 1 * g 1 ( x 1 \_j ) + w 2 * g 2 ( x 2 \_j ) + … wk * gk ( xk\_j ) + wn * gn ( x 3 \_j )$$

In the above Math. 1, “S_Yi_j” denotes a (estimated) stress value of a target subject Yi in unit time (an average, a cumulative total, a difference, max-min, and so on) when data of: x_j (environmental trigger) has been input. “x_j” (={x1_j, x2_j, . . . , xn_j}) denotes a data set of environmental triggers that influence stress during an event, such as a meeting. “f(X1_j, X2_j, . . . , Xn_j)” denotes a model function that calculates a stress value with use of the data set of environmental triggers, x_j, as an input. “g*(x_j)” denotes a function for converting (mapping) a stress value related to the *^{th} element of the data set of environmental triggers, x_j, into a characteristic amount. “w*” denotes a weight related to the *^{th} element of the data set of environmental triggers, x_j.

Regarding the above Math. 1, provided that an activity is a meeting for instance, specific examples of the data set of environmental triggers, x_j, include the places of the meeting, whether a certain person has participated, whether a certain person has made a speech, the amount (time period) of speech made by a certain person, and so on. Therefore, by using the data set of environmental triggers, x_j, and the stress value S_Yi_j corresponding thereto as training data, the weight w* can be learned (optimized) through machine learning and the like.

Also, as new training data can be obtained from a newly calculated stress level and anew activity history, the influence degree calculation unit 13 can update the weight through learning with the addition of the new training data.

Once the weight has been calculated in the foregoing manner, the influence degree calculation unit 13 calculates an influence degree with respect to the specified person in accordance with the set weight. For example, assume that the stress value of the target subject Yi is changing due to a speech made by a person Z. In this case, using the regression coefficient of the regression model, the influence degree calculation unit 13 derives a weight of a trigger related to the attendance of the person Z and a weight of a trigger related to the amount of speech made by the person Z, and multiplies the stress value of the target subject Yi by both of the derived weights. Then, the influence degree calculation unit 13 uses the sum of the obtained products as the influence degree of the person Z with respect to the target subject Yi.

Also, assume that the person specified by the person specification unit 12 is giving stress to a plurality of target subjects. In this case, the influence degree calculation unit 13 calculates the influence degree of this person based on the stress levels of respective target subjects.

Furthermore, the influence degree calculation unit 13 can also set weights respectively for the plurality of target subjects in accordance with the relationships between the person specified by the person specification unit 12 and respective target subjects, and calculate the influence degree of this person based on the weights and the stress levels of respective target subjects. For example, in a case where the target subjects are composed of supervisors and subordinates of the specified person, the influence degree calculation unit 13 sets relatively small weights for the supervisors, and sets relatively large weights for the subordinates.

In the example embodiment, the output unit 14 notifies the specified person of, for example, the influence degree calculated by the influence degree calculation unit 13 as management information. For example, in a case where a terminal device of the specified person exists on the network 40, the output unit 14 transmits an electronic mail or a short message indicating the influence degree to the terminal device of the specified person.

Furthermore, the output unit 14 can also notify a manager who manages the specified person of information of the specified person, which is associated with the calculated influence degree, as management information. For example, in a case where a terminal device of the manager exists on the network 40, the output unit 14 transmits an electronic mail or a short message indicating the influence degree to the terminal device of the manager.

Furthermore, the output unit 14 can also change the destination of notification in accordance with the influence degree. For example, the output unit 14 notifies the terminal device of the specified person in a case where the influence degree of the specified person is equal to or lower than a threshold, and notifies the terminal device of the manager of the specified person in a case where the influence degree of the specified person exceeds the threshold. The output unit 14 can also change the method of notification in accordance with the influence degree.

Also, the destination to which the output unit 14 outputs management information is not limited to the foregoing examples. The output destination of management information may be, for example, an output destination that has been set in advance, such as a family member and a close colleague. Furthermore, the output unit 14 can also output management information to a constituent member of the group to which the specified person belongs.

[Apparatus Operations]

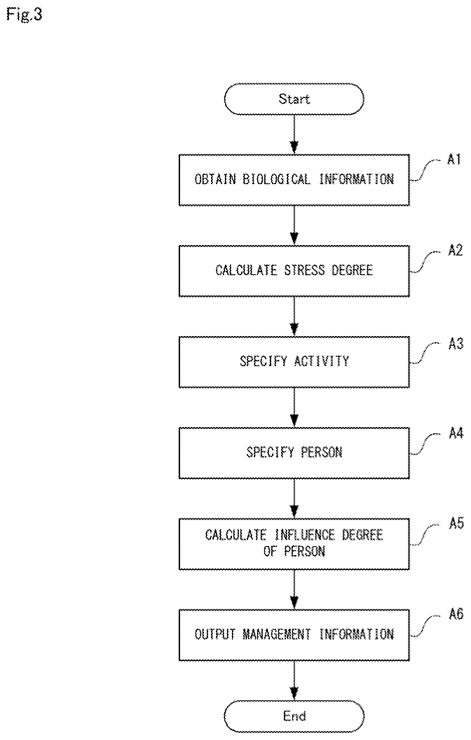

Next, the operations of the stress management apparatus 10 according to the example embodiment will be described using FIG. 3. FIG. 3 is a flow diagram illustrating the operations of the stress management apparatus according to the example embodiment. In the following description, FIG. 1 and FIG. 2 will be referred to as appropriate. Also, in the example embodiment, the stress management method is implemented by causing the stress management apparatus 10 to operate. Therefore, the following description of the operations of the stress management apparatus 10 applies to the stress management method according to the example embodiment.

First, as illustrated in FIG. 3, the physiological information obtainment unit 15 obtains physiological information of each of the target subjects 31a to 31d (step A1). In practice, physiological information of each target subject is obtained via a sensor, a wearable terminal, and the like mounted on the body of the target subject, and imported to a terminal device. Therefore, in step A1, the physiological information obtainment unit 15 obtains pieces of physiological information of respective target subjects from respective terminal devices 30a to 30d via the network 40.

Next, the stress level calculation unit 11 calculates a stress level of each target subject based on the pieces of physiological information of respective target subjects 31a to 31d obtained in step A1 (step A2). In step A2, the stress level is calculated using an existing method in accordance with the type of physiological information.

Next, for each of the target subjects 31a to 31d, the person specification unit 12 specifies an activity that causes stress on the target subject from the activity histories managed in the activity history management apparatus 50 (step A3). For example, in a case where a target subject received an electronic mail in a time period in which the stress level of the target subject exceeded a set value, the person specification unit 12 specifies the reception of the electronic mail as the activity that causes stress. For example, in a case where a target subject attended a meeting in a time period in which the stress level of the target subject exceeded a set value, the person specification unit 12 specifies the attendance at the meeting as the activity that causes stress.

Next, the person specification unit 12 specifies a person involved in the activity specified in step A3 (step A4). For example, in a case where the activity is the reception of an electronic mail, the person specification unit 12 specifies, from the electronic mail that served as the cause, at least one of the receiver, the sender, and people who appear in the text. Also, in a case where the activity is the attendance at a meeting, the person specification unit 12 specifies a person involved in the meeting from information related to the meeting.

Next, for each of the target subjects 31a to 31d, the influence degree calculation unit 13 calculates an influence degree of the person specified in step A4 based on the stress level calculated in step A2 (step A5). In step A5, the influence degree calculation unit 13 can also set a weight for the specified person in accordance with the relationship between the specified person and the target subject, and calculate the influence degree in accordance with the set weight. Also, as stated earlier, the weight is set and updated using, for example, a regression model.

The output unit 14 outputs management information based on the influence degree calculated in step A5 (step A6). In step A6, for example, the output unit 14 notifies the person specified in step A4 of the influence degree calculated in step A5. Furthermore, the output unit 14 can also associate information of the person specified in step A4 with the influence degree calculated in step A5, and notify a manager who manages the person specified in step A4 of the result of the association as management information.

As described above, according to the example embodiment, a person related to the cause of stress is specified from an activity history of a target subject for whom a stress level is to be calculated. Also, the specification of the person is conducted without requiring the person to utter keywords. Therefore, the example embodiment also makes it possible to estimate a stressor who gives stress to another person without giving out specific keywords.

[Program]

It suffices for a program in the example embodiment to be a program that causes a computer to carry out steps A1 to A6 illustrated in FIG. 3. Also, by this program being installed and executed in the computer, the stress management apparatus 10 and the stress management method according to the example embodiment can be realized. In this case, a processor of the computer functions and performs processing as the stress level calculation unit 11, the person specification unit 12, the influence degree calculation unit 13, the output unit 14, and the physiological information obtainment unit 15. The computer includes general-purpose PC, smartphone and tablet-type terminal device.

Furthermore, the program according to the example embodiment may be executed by a computer system constructed with a plurality of computers. In this case, for example, each computer may function as one of the stress level calculation unit 11, the person specification unit 12, the influence degree calculation unit 13, the output unit 14, and the physiological information obtainment unit 15.

[Physical Configuration]

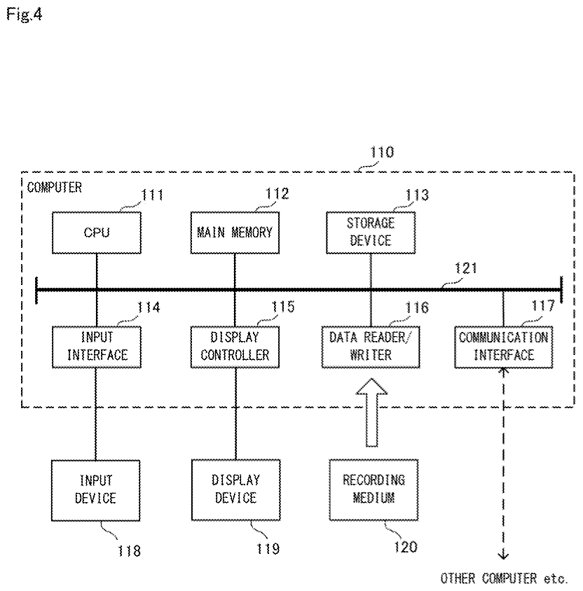

Using FIG. 4, the following describes a computer that realizes the stress management apparatus 10 by executing the program according to the present example embodiment. FIG. 4 is a block diagram illustrating an example of a computer that realizes the stress management apparatus according to the example embodiment.

As shown in FIG. 4, a computer 110 includes a CPU (Central Processing Unit) 111, a main memory 112, a storage device 113, an input interface 114, a display controller 115, a data reader/writer 116, and a communication interface 117. These components are connected in such a manner that they can perform data communication with one another via a bus 121.

The computer 110 may include a GPU (Graphics Processing Unit) or an FPGA (Field-Programmable Gate Array) in addition to the CPU 111, or in place of the CPU 111. In this case, the GPU or the FPGA can execute the programs according to the example embodiment.

The CPU 111 carries out various types of calculation by deploying the program (codes) according to the present example embodiment stored in the storage device 113 to the main memory 112 and executing the codes in a predetermined order. The main memory 112 is typically a volatile storage device, such as a DRAM (dynamic random-access memory).

Also, the program according to the example embodiment is provided in a state where it is stored in a computer-readable recording medium 120. Note that the program according to the present example embodiment may be distributed over the Internet connected via the communication interface 117.

Also, specific examples of the storage device 113 include a hard disk drive and a semiconductor storage device, such as a flash memory. The input interface 114 mediates data transmission between the CPU 111 and an input device 118, such as a keyboard and a mouse. The display controller 115 is connected to a display device 119, and controls display on the display device 119.

The data reader/writer 116 mediates data transmission between the CPU 111 and the recording medium 120, reads out the program from the recording medium 120, and writes the result of processing in the computer 110 to the recording medium 120. The communication interface 117 mediates data transmission between the CPU 111 and another computer.

Specific examples of the recording medium 120 include: a general-purpose semiconductor storage device, such as CF (CompactFlash®) and SD (Secure Digital); a magnetic recording medium, such as a flexible disk; and an optical recording medium, such as a CD-ROM (Compact Disk Read Only Memory).

Note that the stress management apparatus 10 according to the example embodiment can also be realized by using items of hardware that respectively correspond to the components, rather than the computer in which the program is installed. Furthermore, a part of the stress management apparatus 10 may be realized by the program, and the remaining part of the stress management apparatus 10 may be realized by hardware.

A part or an entirety of the above-described example embodiment can be represented by (Supplementary Note 1) to (Supplementary Note 30) described below, but is not limited to the description below.

(Supplementary Note 1)

A stress management apparatus, including:

a stress level calculation unit that calculates a stress level of a target subject based on physiological information of the target subject;

a person specification unit that specifies, based on an activity history of the target subject, a person involved in an activity that has caused stress of the calculated stress level to be incurred;

an influence degree calculation unit that calculates, based on the calculated stress level, an influence degree indicating a degree of influence of the specified person on stress given to the target subject; and

an output unit outputs management information based on the calculated influence degree.

(Supplementary Note 2)

The stress management apparatus according to Supplementary Note 1, wherein

in a case where a plurality of people have been specified, the influence degree calculation unit calculates the influence degree for each of the plurality of people based on the calculated stress level.

(Supplementary Note 3)

The stress management apparatus according to Supplementary Note 1 or 2, wherein

the influence degree calculation unit sets a weight for the specified person in accordance with a relationship between the specified person and the target subject, and calculates the influence degree in accordance with the set weight.

(Supplementary Note 4)

The stress management apparatus according to Supplementary Note 3, wherein

the activity is an activity intended for an electronic mail,

the person specification unit specifies at least one of a receiver and a sender of the electronic mail and people who appear in text thereof as the person, and

the influence degree calculation unit sets a weight for the specified person in accordance with a role of the specified person in the electronic mail, and calculates the influence degree in accordance with the set weight.

(Supplementary Note 5)

The stress management apparatus according to Supplementary Note 3, wherein

the activity is an activity intended for a meeting,

the person specification unit specifies a person involved in the meeting as the person, and

the influence degree calculation unit sets a weight for the specified person in accordance with a behavior of the specified person in the meeting, and calculates the influence degree in accordance with the set weight.

(Supplementary Note 6)

The stress management apparatus according to any one of Supplementary Notes 1 to 5, wherein

in a case where the specified person has been giving stress to a plurality of target subjects, the influence degree calculation unit calculates the influence degree based on a stress level of each of the plurality of target subjects.

(Supplementary Note 7)

The stress management apparatus according to Supplementary Note 6, wherein

the influence degree calculation unit sets a weight for each of the plurality of target subjects in accordance with relationships between the specified person and the plurality of target subjects, and calculates the influence degree based on the weight and the stress level of each of the plurality of target subjects.

(Supplementary Note 8)

The stress management apparatus according to any one of Supplementary Notes 1 to 7, wherein

the output unit notifies the specified person of the calculated influence degree as the management information.

(Supplementary Note 9)

The stress management apparatus according to any one of Supplementary Notes 1 to 8, wherein

the output unit notifies a manager who manages the specified person of information of the specified person as the management information, the information of the specified person being associated with the calculated influence degree.

(Supplementary Note 10)

The stress management apparatus according to any one of Supplementary Notes 1 to 9, wherein

the output unit changes a notification destination of the management information in accordance with a value of the calculated influence degree.

(Supplementary Note 11)

A stress management method, including:

a stress level calculation step of calculating a stress level of a target subject based on physiological information of the target subject;

a person specification step of specifying, based on an activity history of the target subject, a person involved in an activity that has caused stress of the calculated stress level to be incurred;

an influence degree calculation step calculating, based on the calculated stress level, an influence degree indicating a degree of influence of the specified person on stress given to the target subject; and

an output step of outputting management information based on the calculated influence degree.

(Supplementary Note 12)

The stress management method according to Supplementary Note 11, wherein

in the influence degree calculation step, in a case where a plurality of people have been specified, the influence degree is calculated for each of the plurality of people based on the calculated stress level.

(Supplementary Note 13)

The stress management method according to Supplementary Note 11 or 12, wherein

in the influence degree calculation step, a weight is set for the specified person in accordance with a relationship between the specified person and the target subject, and the influence degree is calculated in accordance with the set weight.

(Supplementary Note 14)

The stress management method according to Supplementary Note 13, wherein

the activity is an activity intended for an electronic mail,

in the person specification step, at least one of a receiver and a sender of the electronic mail and people who appear in text thereof is specified as the person, and

in the influence degree calculation step, a weight is set for the specified person in accordance with a role of the specified person in the electronic mail, and the influence degree is calculated in accordance with the set weight.

(Supplementary Note 15)

The stress management method according to Supplementary Note 13, wherein

the activity is an activity intended for a meeting,

in the person specification step, a person involved in the meeting is specified as the person, and

in the influence degree calculation step, a weight is set for the specified person in accordance with a behavior of the specified person in the meeting, and the influence degree is calculated in accordance with the set weight.

(Supplementary Note 16)

The stress management method according to any one of Supplementary Notes 11 to 15, wherein

in the influence degree calculation step, in a case where the specified person has been giving stress to a plurality of target subjects, the influence degree is calculated based on a stress level of each of the plurality of target subjects.

(Supplementary Note 17)

The stress management method according to Supplementary Note 16, wherein

in the influence degree calculation step, a weight is set for each of the plurality of target subjects in accordance with relationships between the specified person and the plurality of target subjects, and the influence degree is calculated based on the weight and the stress level of each of the plurality of target subjects.

(Supplementary Note 18)

The stress management method according to any one of Supplementary Notes 11 to 17, wherein

in the output step, the specified person is notified of the calculated influence degree as the management information.

(Supplementary Note 19)

The stress management method according to any one of Supplementary Notes 11 to 18, wherein

in the output step, a manager who manages the specified person is notified of information of the specified person as the management information, the information of the specified person being associated with the calculated influence degree.

(Supplementary Note 20)

The stress management method according to any one of Supplementary Notes 11 to 19, wherein

in the output step, a notification destination of the management information is changed in accordance with a value of the calculated influence degree.

(Supplementary Note 21)

A computer readable recording medium that includes a program recorded thereon, the program including instructions that cause a computer to carry out:

a stress level calculation step of calculating a stress level of a target subject based on physiological information of the target subject;

a person specification step of specifying, based on an activity history of the target subject, a person involved in an activity that has caused stress of the calculated stress level to be incurred;

an influence degree calculation step of calculating, based on the calculated stress level, an influence degree indicating a degree of influence of the specified person on stress given to the target subject; and

an output step of outputting management information based on the calculated influence degree.

(Supplementary Note 22)

The computer readable recording medium according to Supplementary Note 21, wherein

in the influence degree calculation step, in a case where a plurality of people have been specified, the influence degree is calculated for each of the plurality of people based on the calculated stress level.

(Supplementary Note 23)

The computer readable recording medium according to Supplementary Note 21 or 22, wherein

in the influence degree calculation step, a weight is set for the specified person in accordance with a relationship between the specified person and the target subject, and the influence degree is calculated in accordance with the set weight.

(Supplementary Note 24)

The computer readable recording medium according to Supplementary Note 23, wherein

the activity is an activity intended for an electronic mail,

in the person specification step, at least one of a receiver and a sender of the electronic mail and people who appear in text thereof is specified as the person, and

in the influence degree calculation step, a weight is set for the specified person in accordance with a role of the specified person in the electronic mail, and the influence degree is calculated in accordance with the set weight.

(Supplementary Note 25)

The computer readable recording medium according to Supplementary Note 23, wherein

the activity is an activity intended for a meeting,

in the person specification step, a person involved in the meeting is specified as the person, and

in the influence degree calculation step, a weight is set for the specified person in accordance with a behavior of the specified person in the meeting, and the influence degree is calculated in accordance with the set weight.

(Supplementary Note 26)

The computer readable recording medium according to any one of Supplementary Notes 21 to 25, wherein

in the influence degree calculation step, in a case where the specified person has been giving stress to a plurality of target subjects, the influence degree is calculated based on a stress level of each of the plurality of target subjects.

(Supplementary Note 27)

The computer readable recording medium according to Supplementary Note 26, wherein

in the influence degree calculation step, a weight is set for each of the plurality of target subjects in accordance with relationships between the specified person and the plurality of target subjects, and the influence degree is calculated based on the weight and the stress level of each of the plurality of target subjects.

(Supplementary Note 28)

The computer readable recording medium according to any one of Supplementary Notes 21 to 27, wherein

in the output step, the specified person is notified of the calculated influence degree as the management information.

(Supplementary Note 29)

The computer readable recording medium according to any one of Supplementary Notes 21 to 28, wherein

in the output step, a manager who manages the specified person is notified of information of the specified person as the management information, the information of the specified person being associated with the calculated influence degree.

(Supplementary Note 30)

The computer readable recording medium according to any one of Supplementary Notes 21 to 29, wherein

in the output step, a notification destination of the management information is changed in accordance with a value of the calculated influence degree.

Although the invention of the present application has been described above with reference to the example embodiment, the invention of the present application is not limited to the above-described example embodiment. Various changes that can be understood by a person skilled in the art within the scope of the invention of the present application can be made to the configuration and the details of the invention of the present application.

### INDUSTRIAL APPLICABILITY

As described above, according to the present invention, it is possible to predict the future state of stress of the user. The present invention is useful for human stress management system.

### REFERENCE SIGNS LIST

10 Stress management apparatus
11 Stress level calculation unit
12 Person specification unit
13 Influence degree calculation unit
14 Output unit
15 Physiological information obtainment unit
30a-30d Terminal device
31a-31d Target subject
40 Network
50 Activity history management apparatus
110 Computer
111 CPU
112 Main memory
113 Storage device
114 Input interface
115 Display controller
116 Data reader/writer
117 Communication interface
118 Input device
119 Display device
120 Recording medium
121 Bus

What is claimed is:

1. A stress management apparatus, comprising:
at least one memory storing instructions; and
at least one processor configured to execute the instructions to:
calculate a stress level of a target subject based on physiological information of the target subject;
specify, based on an activity history of the target subject, a person involved in an activity that has caused stress of the calculated stress level to be incurred;
calculate, based on the calculated stress level, an influence degree indicating a degree of influence of the specified person on stress given to the target subject; and
output management information based on the calculated influence degree.

2. The stress management apparatus according to claim 1, wherein further at least one processor configured to execute the instructions to:
in a case where a plurality of people have been specified, calculate the influence degree for each of the plurality of people based on the calculated stress level.

3. The stress management apparatus according to claim 1, wherein further at least one processor configured to execute the instructions to:
set a weight for the specified person in accordance with a relationship between the specified person and the target subject, and calculates the influence degree in accordance with the set weight.

4. The stress management apparatus according to claim 3, wherein
the activity is an activity intended for an electronic mail,
further at least one processor configured to execute the instructions to:
specify at least one of a receiver and a sender of the electronic mail and people who appear in text thereof as the person, and
set a weight for the specified person in accordance with a role of the specified person in the electronic mail, and calculates the influence degree in accordance with the set weight.

5. The stress management apparatus according to claim 3, wherein
the activity is an activity intended for a meeting,
the person specification means specifies a person involved in the meeting as the person, and
further at least one processor configured to execute the instructions to:
set a weight for the specified person in accordance with a behavior of the specified person in the meeting, and calculates the influence degree in accordance with the set weight.

6. The stress management apparatus according to claim 1, wherein further at least one processor configured to execute the instructions to:
calculate, in a case where the specified person has been giving stress to a plurality of target subjects, the influence degree based on a stress level of each of the plurality of target subjects.

7. The stress management apparatus according to claim 6, wherein further at least one processor configured to execute the instructions to:
set a weight for each of the plurality of target subjects in accordance with relationships between the specified person and the plurality of target subjects, and calculate the influence degree based on the weight and the stress level of each of the plurality of target subjects.

8. The stress management apparatus according to claim 1, wherein further at least one processor configured to execute the instructions to:
notify the specified person of the calculated influence degree as the management information.

9. The stress management apparatus according to claim 1, wherein further at least one processor configured to execute the instructions to:
notify a manager who manages the specified person of information of the specified person as the management information, the information of the specified person being associated with the calculated influence degree.

10. The stress management apparatus according to claim 1, wherein further at least one processor configured to execute the instructions to:
change a notification destination of the management information in accordance with a value of the calculated influence degree.

11. A stress management method, comprising:
calculating a stress level of a target subject based on physiological information of the target subject;
specifying, based on an activity history of the target subject, a person involved in an activity that has caused stress of the calculated stress level to be incurred;
calculating, based on the calculated stress level, an influence degree indicating a degree of influence of the specified person on stress given to the target subject; and
outputting management information based on the calculated influence degree.

12.-20. (canceled)

21. A non-transitory computer readable recording medium that includes a program recorded thereon, the program including instructions that cause a computer to:
calculate a stress level of a target subject based on physiological information of the target subject;
based on an activity history of the target subject, specify a person involved in an activity that has caused stress of the calculated stress level to be incurred;
based on the calculated stress level, calculate an influence degree indicating a degree of influence of the specified person on stress given to the target subject; and
output management information based on the calculated influence degree.

22.-30. (canceled)
